(19) United States
(12) Patent Application Publication
(10) Pub. No.: US 2021/0052368 A1
(43) Pub. Date: Feb. 25, 2021

(54) LENS SYSTEMS FOR VISUAL CORRECTION AND ENHANCEMENT

(71) Applicants: David SMADJA, Tel Aviv (IL); BAR ILAN UNIVERSITY, Ramat-Gan (IL)

(72) Inventors: David SMADJA, Tel Aviv (IL); Zeev ZALEVSKY, Rosh Haayin (IL)

(21) Appl. No.: 16/961,968

(22) PCT Filed: Jan. 14, 2019
(86) PCT No.: PCT/IL2019/050053

§ 371 Date: Jul. 14, 2020

Related U.S. Application Data
(60) Provisional application No. 62/617,256, filed on Jan. 14, 2018.

(51) Int. Cl. A61F 2/16; G02F 1/01; G02C 7/10; G02C 11/04

(52) U.S. Cl. CPC A61F 2/16; G02F 1/0126; G02F 2202/10; G02C 11/04; G02C 7/10

(57) ABSTRACT

A system for vision correction and enhancement which may include lenses and surfaces coated with materials with tunable reflectivity is provided. Methods of using the system for correcting and enhancing vision is also provided.

### CROSS-REFERENCE TO RELATED APPLICATIONS

This application claims the benefit of priority of U.S. Provisional Patent Application No. 62/617,256, titled “LENS SYSTEMS FOR VISUAL CORRECTION AND ENHANCEMENT” filed on Jan. 14, 2018. The contents of the above applications are all incorporated by reference as if fully set forth herein in their entirety.

### FIELD OF INVENTION

The present invention is in the field of optics and vison correction.

### BACKGROUND OF THE INVENTION

The World Health Organization (WHO) estimated in 2010 that 285 million people are visually impaired worldwide. Of those, approximately 246 million have low vision or some partial vision impairment. WHO further estimated that some 80% of all visual impairment can be prevented or cured, and that the leading cause of visual impairment is uncorrected refractive errors.

Presbyopia, the normal loss of near focusing ability that occurs with age is one of the most prevalent vision disorders. In the United States alone 112 million Americans were presbyopic in 2006, and that number is expected to increase to over 123 million by 2020. Astigmatism, nearsightedness and farsightedness are also common forms of corrective refractive errors. Even with the growth of corrective laser eye surgery the most prevalent form of vision correction in the world is still eye glasses and/or contact lenses.

People suffering from presbyopia and far-sightedness have difficulty in seeing from a close distance. Such difficulties are particularly troublesome in industrial countries with high literacy levels, and high computer use. Reading, either from a book or a screen, is affected in those suffering from presbyopia and far-sightedness, and activities taken for granted by those with healthy vision, such as reading a text or email, can be impaired. Corrective lenses, such as bifocals, trifocals and progressive lenses are the most common solutions for this form of visual impairment.

These types of corrective lenses have glass with different reflectivity in different parts of the lens. Bifocals will have two types of glass one on the upper half (far-distance) and one on the bottom (near-distance) that will give different corrections for different distances. Trifocals will have three different pieces of glass, and progressives will have many different areas that allow for focuses as multiple distances. The major drawback however or all these devices is that it requires the user to look only with a portion of the glass. This leads to a restricted field of vision and frequently eye strain. Further, as the eye passes from one region to another, a disturbing double image can be seen, and incorrect reflective images can also occur. Progressive lenses also have dead areas on the periphery where no correction is achieved.

Owing to the annoyance and appearance of eyeglasses, contact lenses and laser surgery are growing in popularity. However, both have major drawbacks in efficiency and cost. Laser surgery is costly, not always successful and frequently requires further correction later in life. Contact lenses, either require continuous purchase of more lenses or the use of harder more uncomfortable lenses. Though bifocal, trifocal and progressive contacts exist, they are even more expensive and suffer from the same problems as their eye glasses counterparts. A corrective vision system that does not reduce the field of vision, does not create false reflective images and can be easily modified as one's vision changes is greatly needed.

### SUMMARY OF INVENTION

The following embodiments and aspects thereof are described and illustrated in conjunction with systems, tools and methods which are meant to be exemplary and illustrative, not limiting in scope. According to an aspect, there is provided a lens system including a first lens and a second lens arranged coaxially along a central axis, the central axis passing through vertices of the first and second lenses, and a plurality of surfaces arranged along the central axis, wherein (a) at least the second lens is sandwiched between two of the surfaces, at least one of the surfaces being at least partially coated with a first semiconducting material with tunable-reflectivity, and (b) at least two of the surfaces are at least partially coated with a second semiconducting material with tunable-reflectivity and define between them a free space optical path.

According to some embodiments, the first and second semiconducting materials with tunable-reflectivity are the same.

According to an aspect, there is provided a lens system comprising a first lens, a free space optical path and a second lens located between the first lens and the free space optical path, the lenses and optical path placed concentrically along a central axis, wherein the central axis passes through a vertex of the first lens and through a vertex of the second lens, and wherein the second lens is between surfaces at least partially coated with a first semiconducting material with tunable-reflectivity, and the free space optical path is between surfaces coated with a second semiconducting material with tunable-reflectivity.

In some embodiments, the surfaces are oriented generally parallel to the equator of one or more of the lenses. In some embodiments, the surfaces are oriented generally perpendicular to the central axis.

According to some embodiments, the distance between the first lens and the second lens is no more than 100 mm. According to some embodiments, the free space optical path extends along the central axis between 1 and 100 mm. According to some embodiments, a central beam of the free space optical path coincides with and extends along the central axis.

According to some embodiments, the first lens has a thickness of between 0.1 and 10 mm. According to some embodiments, the second lens has a thickness of between 0.1 and 10 mm.

According to some embodiments, the coating with a first semiconducting material with tunable-reflectivity faces the second lens. According to some embodiments, the coating with a second semiconducting material with tunable-reflectivity faces the interior of the free space optical path. In some embodiments, the coating comprises at least a portion of one or both surfaces of one or more of the lenses.

According to some embodiments, light entering the free space optical path is reflected between coated surfaces defining the free space optical path. In some embodiments, light entering the free space optical path is reciprocally reflected between coated surfaces defining the free space optical path. According to some embodiments, the reflecting creates a resonator between the coated surfaces of the region.

According to some embodiments, at least one of the semiconducting materials is tunable by contact with a laser or LED light. According to some embodiments, the laser or LED light's wavelength is not greater than 400 nm. According to some embodiments, the first semiconducting material is tunable by contact with a first laser or LED light and the second semi conducting material is tunable by contact with a second laser or LED, and wherein the first laser and the second laser have different wavelengths. According to some embodiments, the different wavelengths are not higher than 400 nm.

According to some embodiments, the first or second semiconducting material is selected from the group consisting of: semiconductors absorbing at the desired wavelength, semiconductors with synthesized or engineered bandgaps allowing enhanced absorption at the desired wavelength, and a surface having plasmonic nanostructures to enhance the surface light absorption process at the desired wavelength. According to some embodiments, the first or second semiconducting material is aluminum nitride.

According to some embodiments of the invention, the lens system is configured for interocular insertion. According to some embodiments, interocular insertion is about 17 mm from the retina.

According to some embodiments, the lens system is configured to correct a defect selected from a group consisting of: myopia, hyperopia, presbyopia, cataracts, macular degeneration, retinal neuropathy and glaucoma.

According to some embodiments, the lens system of the invention is for use in enhancing or amplifying vision. According to some embodiments, the lens system of the invention is for use in optical zooming.

According to some embodiments, the lens system of the invention further comprises a light source adapted to tune the reflectivity of at least one of the materials with tunable reflectivity. According to some embodiments, the light source is a laser or LED light.

According to an aspect, there is provided a vision correction and enhancement system, comprising:
a. any one of the lens systems of the invention; and
b. at least one laser diode capable of producing laser or LED light at at-least one wavelength capable of tuning the reflectivity of at least one of the semiconducting materials.

According to some embodiments, the laser diode or LED is mounted on glasses and configured to shine laser light on the lens system. According to some embodiments, the glasses are configured to block light at or near the wavelength of the laser light produced by the laser diode or LED. According to some embodiments, the laser diode or LED is capable of producing external excitation light at a plurality of wavelengths capable of tuning the reflectivity of the first and the second semiconducting materials.

According to an aspect, there is provided a method of correcting or enhancing vision in a subject in need thereof, the method comprising inserting into an eye of the subject a lens system of the invention.

According to some embodiments, the inserting is performed during cataract or lens replacement surgery.

According to some embodiments, the method further comprises providing to the subject at least one laser diode capable of producing laser or LED light at at-least one wavelength capable of tuning the reflectivity of at least one of the semiconducting materials of the lens system.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a block diagram illustrating a lens system according to some embodiments of the invention;

FIG. 2A illustrates an operation of a lens system according to embodiments of the invention;

FIG. 2B illustrates an operation of a lens system according to embodiments of the invention;

FIG. 2C illustrates an operation of a lens system according to embodiments of the invention;

FIG. 2D illustrates an operation of a lens system according to embodiments of the invention;

FIG. 3A is a photograph showing results of the use of an embodiment of the lens system for looking at a faraway object;

FIG. 3B is a photograph showing results of the use of an embodiment of the lens system for looking at a close object; and

FIG. 3C is a photograph showing results of the use of an embodiment of the lens system for magnification.

### DETAILED DESCRIPTION

The present invention, in some embodiments thereof, relates to lens systems, vision correction and enhancement systems and methods of vision correction and enhancement. In particular, the invention discloses an arrangement of lenses and optical paths coated with one or more materials with tunable reflectivity that can be controlled by laser or LED light.

As used herein, the term “vision correction” refers to improving blurred, out of focus or distorted vision caused by refractive error or damage to the eye. Examples of refractive error include, but are not limited to, myopia (nearsightedness), hyperopia (farsightedness), astigmatism (malformation of the cornea or lens), and presbyopia (age related myopia). Examples of damage to the eye include, but are not limited to, cataracts, physical injury, macular degeneration, glaucoma and retinal neuropathy.

As used herein, the term “vision enhancement” refers to providing vision capabilities beyond that of a normal healthy eye. In some embodiments, normal vision refers to 20/20 vision (seeing an image at 20 feet clearly as others see it at 20 feet). In some embodiments, enhanced vision comprises optical zooming. In some embodiments, enhanced vision comprises enlarging an image. In some embodiments, enhanced vision comprises providing a person with vision better than 20/20, such as at least 20/15, 20/10, 20/5 or 20/1. Each possibility represents a separate embodiment of the invention. In some embodiments, enhanced vision comprises magnification. In some embodiments, the magnification is at least a 1.5×, 2×, 2.5×, 3×, 3.5×, 4×, 4.5× or 5× magnification. Each possibility represents a separate embodiment of the invention. In some embodiments, the magnification is about a 1.5× magnification. In some embodiments, the magnification is about a 3× magnification. In some embodiments, enhanced vision comprises multiple magnifications. Multiple magnification refers to the ability to magnify what is being seen, by more than one factor, such as, but not limited to, by either 1.25×, 1.5× or 2×.

As used herein, the term “tunable-reflectivity” refers to the quality of being able to change reflectivity in a controlled manner. Thus, a material with tunable-reflectivity is a material whose reflectivity can be changed in a controlled manner, e.g., by shining on it laser or LED light. In some embodiments, the change in reflectivity is from negligible reflectivity to being reflective. In some embodiments, the change in reflectivity is an increase or a decrease in reflectivity. In some embodiments, reflectivity is increased by absorption of photons and generation of free carriers. In some embodiments, the change in reflectivity is proportionate to the wavelength of the light.

In some embodiments, the material with tunable reflectivity is a natural material. In some embodiments, the material with tunable reflectivity is a man-made material. In some embodiments, the material with tunable reflectivity is a composite material. In some embodiments, the material with tunable reflectivity is naturally tunable. In some embodiments, the material with tunable reflectivity undergoes modification to make it tunable.

In some embodiments, the increase is at least a 5, 10, 15, 20, 25, 30, 40, 50, 60 70, 80, 90, 95, 100, 150, 200, 250 300, 350, 400, 450 or 500% increase. Each possibility represents a separate embodiment of the invention. In some embodiments, a decrease is at least a 5, 10, 15, 20, 25, 30, 40, 50, 60 70, 80, 90, 95, 97, 99 or 100% decrease. Each possibility represents a separate embodiment of the invention.

As used herein, the term “light” refers to any electromagnetic radiation. In some embodiments, light refers to visible light. In some embodiments, light refers to light visible to a human. In some embodiments, light refers to photonic light. In some embodiments, light refers to ultra violet light.

### Lens System

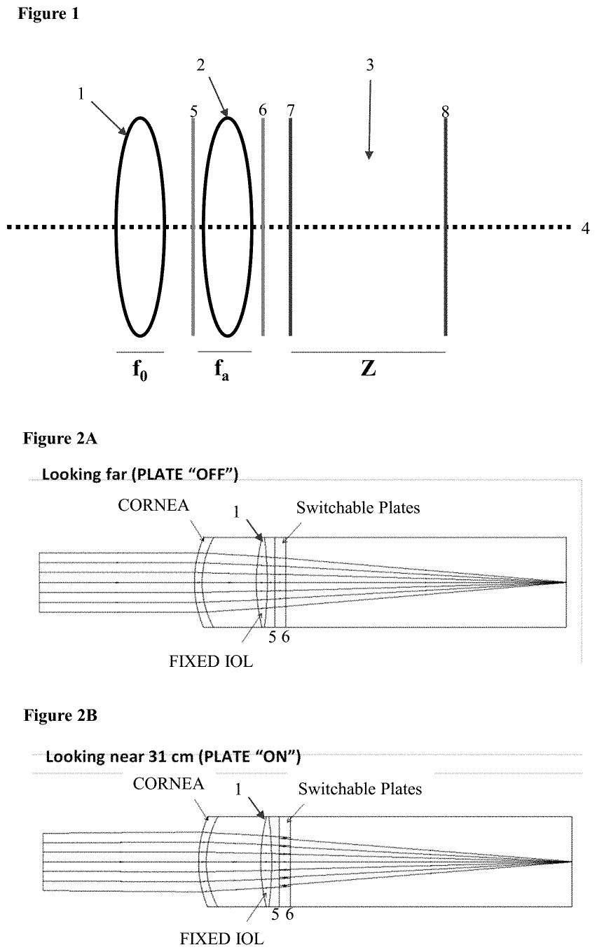

Reference is made to FIG. 1 showing a lens system according to illustrative embodiments of the present invention. As shown, the system may include a first lens (1), a second lens (2), a free space optical path (3). Additionally, the system may include at least one surface coated with or made of a semiconducting material with tunable-reflectivity (5-8). Disclosed herein is a lens system comprising a first lens (1), a second lens (2) and a free space optical path (3) that all lie one after the other along a central axis (4). In some embodiments, the second lens (2) and/or free space optical path (3) are within regions defined by one or more surfaces coated with semiconducting materials with tunable-reflectivity. In some embodiments, the coating comprises at least a portion of one or both surfaces of one or more of the lenses.

In some embodiments, the regions are bound by one or more surfaces of the semiconducting material. In some embodiments, the system does not comprise the first lens (1). In some embodiments, the system comprises more than two lenses. These further lenses may be sandwiched by coated surfaces or may be uncoated. In some embodiments, one or more surfaces of one or more of the lenses faces coated surfaces. In some embodiments, at least a portion of one or more surfaces of one or more of the lenses comprises a coated surface. In some embodiments, the second lens (2) is between two surfaces (5-6) coated with semiconducting materials with tunable reflectivity. In some embodiments, the second lens has on one side a surface (5 or 6) coated with semiconducting materials with tunable reflectivity. In some embodiments, the surface is between the first lens and second lens. In some embodiments, the surface is between the second lens and the free space optical path. In some embodiments, the free space optical path (4) is placed or located between two surfaces (7-8) coated with semiconducting materials with tunable reflectivity. In some embodiments, the central axis (4) passes through a vertex of the first (1) and/or second (2) lenses. In some embodiments, the second lens (2) is between two surfaces coated with a first semiconducting material with tunable-reflectivity (5 and 6), and the free space optical path (3) is placed or located between two surfaces coated with a second semiconducting material with tunable-reflectivity (7 and 8). In some embodiments, the first and second semiconducting material with tunable-reflectivity are the same material. In some embodiments, the first and second semiconducting material with tunable-reflectivity are different materials. As used herein, “to tune” refers to changing the reflectivity of a material. In some embodiments, the first and second semiconducting material with tunable-reflectivity are tunable with different wavelengths of light.

In some embodiments, the coatings of first semiconducting materials and coatings of second semiconducting materials face each other such that a light wave or photon would reflect between the two coatings. In some embodiments, the coatings of the first semiconducting materials (5 and 6) face each other such that a light wave or photon would reflect between the two coatings. In some embodiments, the coatings of the second semiconducting materials (7 and 8) face each other such that a light wave or photon would reflect between the two coatings. In some embodiments, the coating of a surface (5) faces toward the second lens (2). In some embodiments, the coating of a surface (6) faces toward the second lens (2). In some embodiments, the coating of a surface (5) faces away from the second lens (2). In some embodiments, the coating of a surface (6) faces away from the second lens. In some embodiments, the second lens (2) is directly coated itself. In some embodiments, a surface is coated on both sides. In some embodiments, the second lens (2) is within a region that is coated. In some embodiments, the second lens (2) is between two surfaces (5-6) that are coated. In some embodiments, light reflection between two coated surfaces creates a resonator between the coated surfaces of a region.

Examples of methods for coating surfaces include, but are not limited to spray coating, thermal spraying, electroplating, sherardizing, hot-dip galvanizing, nan-coating, and liquid glass coating. Materials such as glass may be coated differently than a metal or ceramic. In some embodiments, the surface is not coated but rather is made of the material with tunable reflectivity. In some embodiments, glass is coated with the material with tunable reflectivity. In some embodiments, metal is coated with the material with tunable reflectivity. In some embodiments, the surface coated with a material of tunable reflectivity allows 100% of light to pass through.

In some embodiments, a semiconducting material, or a surface coated therein allows about 100% of light to pass through them. In some embodiments, once tuned by LED or laser light the semiconducting materials allow less than 100% of light to pass through. In some embodiments, a tuned semiconducting material, or a surface coated therein, allows less than 95%, 90%, 85%, 80%, 75%, 70%, 65%, 60%, 55%, 50%, 45%, 40%, 35%, 30%, 25%, 20%, 15%, 10%, or 5% of light to pass through. Each possibility represents a separate embodiment of the invention. In some embodiments, once tuned by LED or laser light the semiconducting materials allow less than 100%, 99%, 97%, 95%, 90%, 85%, 80%, 75%, 70%, 65%, 60%, 55%, 50%, 45%, 40%, 35%, 30%, 25%, 20%, 15%, 10%, or 5% of light to pass through. Each possibility represents a separate embodiment of the invention.

In some embodiments, the surfaces coated with a semiconducting material on one side are coated on the opposite side with a non-reflective material. In some embodiments, light should pass into the areas between surfaces coated with a semiconducting material and then can be reflected back between the surfaces coated by the semiconducting material. The number of times light is reflected back may be 0, 1, 2, or more.

In some embodiments, reflectivity (R) is calculated using the following formula:

Finesse (F)=π sqrt(R)/(1−R) (1)

As used herein, “finesse” is to be understood by its simple meaning and refers to an optical resonator's (cavity's) free spectral range divided by the full width at half-maximum bandwidth of the resonances. It is a dimensionless measure and can be used to determine the reflectivity and power of the resonator. Finesse is also the number of times the optical rays on average bounced forth and back in the resonator and it is the factor by which one has more photons inside the resonator in respect to outside if continuous illumination is applied and the resonator acts as an optical capacitor collecting and storing photons. In some embodiments, the property of Finesse associated with the invention disclosed herein is the number of times the optical rays travel forth and back between the mirrors of the resonator.

It will be understood that if the focal length of the first lens (1) is f₀ and the focal length of the second lens (2) is fₐ then when light is reflected back between the materials coating the area around the second lens (2) it will have a total focal length (fₘ) of: 1/fₘ=1/f₀+m/fₐ, where m equals the number of times the light passes through the area between the coated surfaces (5 and 6). In some embodiments, the light passes through the second lens (1) once, three times (one reflection) or five times (two reflections).

In some embodiments, the distance between the first and second lenses is not more than 100 mm, 50 mm, 25 mm, 10 mm, 1 mm, 0.5 mm, 400 μm, 300 μm, 200 μm, 190 μm, 180 μm, 170 μm, 160 μm, 150 μm, 140 μm, 130 μm, 120 μm, 110 μm, 100 μm, 90 μm, 80 μm, 70 μm, 60 μm, 50 μm, 40 μm, 30 μm, 20 μm or 15 μm. Each possibility represents a separate embodiment of the invention. In some embodiments, the distance between the first and second lenses is at most 100 μm. In some embodiments, the distance between the first and second lenses is between 10 μm and 100 mm, 10 μm and 10 mm, 10 μm and 1 mm, 10 μm and 0.5 mm, 10 and 400 μm, 10 and 300 μm, 10 and 200 μm, 10 and 100 μm, 10 and 90 μm, 10 and 80 μm, 15 and 130 μm, 15 and 120 μm, 15 and 115 μm, 15 and 100 μm, 15 and 90 μm, 15 and 80 μm, 20 and 130 μm, 20 and 120 μm, 20 and 120 μm, 20 and 100 μm, 20 and 90 μm, 20 and 80 μm, 25 and 130 μm, 25 and 120 μm, 25 and 125 μm, 25 and 100 μm, 25 and 90 μm, or 25 and 80 μm. Each possibility represents a separate embodiment of the invention.

In some embodiments, the first lens lies to the left of the second lens, as depicted in FIG. 1. In some embodiments, light enters the first lens before entering the second lens. In some embodiments, the first lens lies to the right of the second lens. In some embodiments, light enters the second lens before entering the first lens. In some embodiments, the second lens is between the first lens and the free space optical path. In some embodiments, the first lens is between the second lens and the free space optical path. In some embodiments, the first lens is convex and the second concave. In some embodiments, the first lens is concave and the second convex. In some embodiments, both lenses are convex. In some embodiments, both lenses are concave. In some embodiments, the first lens or the second lens are any one of convex, concave, biconvex, planoconvex, positive meniscus, negative meniscus, planoconcave, biconcave. In some embodiments, the first lens or the second lens are either one of converging and diverging.

In some embodiments, the lens system further comprises additional lenses. In some embodiments, the system comprises at least a third, fourth, fifth, sixth, seventh, eighth, ninth, or tenth lens. Each possibility represents a separate embodiment of the invention. In some embodiments, the system comprises a third lens. In some embodiments, the additional lenses may be positioned between or adjacent to any of the other components of the system. In some embodiments, the additional lenses may be coated or not coated, or within or without of an area which is coated. Each of these possibilities represents a separate embodiment of the invention.

In some embodiments, the first lens has a thickness of at least 0.0001, 0.001, 0.01, 0.05, 0.1, 0.15, 0.2, 0.25, 0.3, 0.35, 0.4, 0.45 or 0.5 mm. Each possibility represents a separate embodiment of the invention. In some embodiments, the first lens has a thickness of at least 0.1 mm. In some embodiments, the first lens has a thickness of at most 2, 2.5, 3, 3.5, 4, 4.5, 5, 5.5, 6, 6.5, 7, 7.5, 8, 8.5, 9, 9.5, 10, 10.5, 11, 11.5, 12, or 12.5 mm. Each possibility represents a separate embodiment of the invention. In some embodiments, the first lens has a thickness of at most 10 mm. In some embodiments, the first lens has a thickness of between 0.01 and 12.5, 0.01 and 12, 0.01 and 11.5, 0.01 and 11, 0.01 and 10.5, 0.01 and 10, 0.01 and 9.5, 0.01 and 9, 0.01 and 8.5, 0.01 and 8, 0.05 and 12.5, 0.05 and 12, 0.05 and 11.5, 0.05 and 11, 0.05 and 10.5, 0.05 and 10, 0.05 and 9.5, 0.05 and 9, 0.05 and 8.5, 0.05 and 8, 0.1 and 12.5, 0.1 and 12, 0.1 and 11.5, 0.1 and 11, 0.1 and 10.5, 0.1 and 10, 0.1 and 9.5, 0.1 and 9, 0.1 and 8.5, 0.1 and 8, 0.15 and 12.5, 0.15 and 12, 0.15 and 11.5, 0.15 and 11, 0.15 and 10.5, 0.15 and 10, 0.15 and 9.5, 0.15 and 9, 0.15 and 8.5, 0.15 and 8, 0.2 and 12.5, 0.2 and 12, 0.2 and 11.5, 0.2 and 11, 0.2 and 10.5, 0.2 and 10, 0.2 and 9.5, 0.2 and 9, 0.2 and 8.5, 0.2 and 8, 0.25 and 12.5, 0.25 and 12, 0.25 and 11.5, 0.25 and 11, 0.25 and 10.5, 0.25 and 10, 0.25 and 9.5, 0.25 and 9, 0.25 and 8.5, 0.25 and 8, 0.3 and 12.5, 0.3 and 12, 0.3 and 11.5, 0.3 and 11, 0.3 and 10.5, 0.3 and 10, 0.3 and 9.5, 0.3 and 9, 0.3 and 8.5, or 0.3 and 8 mm. Each possibility represents a separate embodiment of the invention. In some embodiments, the first lens has a thickness of between 0.1 and 2 mm.

In some embodiments, the second lens has a thickness of at least 0.0001, 0.001, 0.01, 0.05, 0.1, 0.15, 0.2, 0.25, 0.3, 0.35, 0.4, 0.45 or 0.5 mm. Each possibility represents a separate embodiment of the invention. In some embodiments, the second lens has a thickness of at least 0.1 mm. In some embodiments, the second lens has a thickness of at most 2, 2.5, 3, 3.5, 4, 4.5, 5, 5.5, 6, 6.5, 7, 7.5, 8, 8.5, 9, 9.5, 10, 10.5, 11, 11.5, 12, or 12.5 mm. Each possibility represents a separate embodiment of the invention. In some embodiments, the second lens has a thickness of at most 10 mm. In some embodiments, the second lens has a thickness of between 0.01 and 12.5, 0.01 and 12, 0.01 and 11.5, 0.01 and 11, 0.01 and 10.5, 0.01 and 10, 0.01 and 9.5, 0.01 and 9, 0.01 and 8.5, 0.01 and 8, 0.05 and 12.5, 0.05 and 12, 0.05 and 11.5, 0.05 and 11, 0.05 and 10.5, 0.05 and 10, 0.05 and 9.5, 0.05 and 9, 0.05 and 8.5, 0.05 and 8, 0.1 and 12.5, 0.1 and 12, 0.1 and 11.5, 0.1 and 11, 0.1 and 10.5, 0.1 and 10, 0.1 and 9.5, 0.1 and 9, 0.1 and 8.5, 0.1 and 8, 0.15 and 12.5, 0.15 and 12, 0.15 and 11.5, 0.15 and 11, 0.15 and 10.5, 0.15 and 10, 0.15 and 9.5, 0.15 and 9, 0.15 and 8.5, 0.15 and 8, 0.2 and 12.5, 0.2 and 12, 0.2 and 11.5, 0.2 and 11, 0.2 and 10.5, 0.2 and 10, 0.2 and 9.5, 0.2 and 9, 0.2 and 8.5, 0.2 and 8, 0.25 and 12.5, 0.25 and 12, 0.25 and 11.5, 0.25 and 11, 0.25 and 10.5, 0.25 and 10, 0.25 and 9.5, 0.25 and 9, 0.25 and 8.5, 0.25 and 8, 0.3 and 12.5, 0.3 and 12, 0.3 and 11.5, 0.3 and 11, 0.3 and 10.5, 0.3 and 10, 0.3 and 9.5, 0.3 and 9, 0.3 and 8.5, or 0.3 and 8 mm. Each possibility represents a separate embodiment of the invention. In some embodiments, the second lens has a thickness of between 0.1 and 2 mm.

In some embodiments, the free space optical path is a rectangular block. In some embodiments, the free space optical path is made of glass or any non-refracting material. In some embodiments, the free space optical path comprises walls and is hollow. In some embodiments, the free space optical path is solid. In some embodiments, the free space optical path extends along the central axis at least 0.0001, 0.001, 0.01, 0.1, 1, 2, 3, 4, 5, 10, 15, 20, 25, 30, 35, 40, 45, 50, 55, 60, 65, 70, 75, 80, 85, 90, 95, or 100 mm. Each possibility represents a separate embodiment of the invention. In some embodiments, the free space optical path extends along the central axis at most 0.001, 0.01, 0.1, 1, 2, 3, 4, 5, 10, 15, 20, 25, 30, 35, 40, 45, 50, 55, 60, 65, 70, 75, 80, 85, 90, 95, or 100 mm. Each possibility represents a separate embodiment of the invention. In some embodiments, the free space optical path extends along the central axis between 0.001 and 2 mm.

In some embodiments, light entering the free space optical path is reflected between the coated surfaces of the path. In some embodiments, this reflecting creates a resonator between the coated surfaces of the path. In some embodiments, the resonator increases the optical length by two times or 4 times the length of the free space optical path (Z, in FIG. 1). In some embodiments, the resonator increases the focal length by two times or 4 times the length of the free space optical path (Z, in FIG. 1). In some embodiments, the resonator increases the focal length and/or the optical length by a multiple of 2. In some embodiments, the increase is by 2, 4, 6, 8, 10, 12 or 14 times. Each possibility represents a separate embodiment of the invention.

### Tunability

In some embodiments, at least one of the semiconducting materials or coated surfaces is tunable by contact with or exposure to laser light. In some embodiments, at least one of the semiconducting materials or surfaces is tunable by contact with a LED light. In some embodiments, at least one of the semiconducting materials or surfaces is tunable by contact with a laser or LED light. In some embodiments, the first and second semiconducting materials are tunable by contact with a laser or LED light. In some embodiments, contact comprises shining the laser or LED light on the material or surface.

In some embodiments, a material or surface with tunable reflectivity possesses the ability to be in at least two states of reflectivity, a basal reflectivity and an excited state of reflectivity. It will be understood that the basal state refers to the natural reflectivity of the material without excitation by a light source. The excited state of reflectivity, therefore, refers to the reflectivity of the material or surface while it is being excited by a light source. In some embodiments, the material or surface is in an excited state of reflectivity when contacted by a laser or LED light. In some embodiments, a tunable material is in a state of excited reflectivity for as long as light of the desired wavelength is in contact with the material. Thus, when the light is shut off or blocked the material or surface will return to its unexcited (basal) reflectivity. In some embodiments, a material or surface may possess more than one state of excited reflectivity. In such embodiments, more than one wavelength is capable of exciting the material or surface. In some embodiments, each wavelength induces a different reflectivity in the material or surface.

In some embodiments, the first material or surface and the second material or surface are excited by the same wavelength. In some embodiments, the first material or surface and the second material or surface are excited by different wavelengths. In some embodiments, the two surfaces (5 and 6) around the second lens (2) are excited by the same wavelength. In some embodiments, the two surfaces (7 and 8) around the free space optical path (3) are excited by the same wavelength.

In some embodiments, the laser or LED light used to tune the reflectivity of coated surfaces 5, 6, 7 and/or 8, has a wavelength below the visible spectrum. In some embodiments, the laser or LED light has a wavelength below 400 nm. In some embodiments, the first and/or second material are tunable by light with a wavelength below 400 nm. It will be understood, that the wavelength of light selected will match the wavelength that can tune the reflectivity of one of the materials. In some embodiments, the laser or LED light used has a wavelength in at least one of the green spectrum of visible light and the blue spectrum of visible light. In some embodiments, the laser or LED light used has a wavelength in the green spectrum. In some embodiments, the laser or LED light used has a wavelength in the blue spectrum. In some embodiments, the laser or LED light used has a wavelength in the ultra violet spectrum. In some embodiments, the laser or LED light has a wavelength below 410, 420, 430, 440, 450, 460, 470, 480, 490, 495, 500, 510, 520, 530, 540, 550, 560, or 570 nm. Each possibility represents a separate embodiment of the invention.

In some embodiments, the first and/or second semiconducting material may be selected from: semiconductors absorbing at the desired wavelength, semiconductors with synthesized or engineered bandgaps allowing enhanced absorption at the desired wavelength, and a surface having plasmonic nanostructures to enhance the surface light absorption process at the desired wavelength. Examples of tunable materials include, but are not limited to aluminum nitride, vanadium dioxide, graphene and other transition metal oxides. In some embodiments, the first or second semiconducting material is aluminum nitride.

### Interocular Insertion and Use

In some embodiments, the lens systems of the invention are configured for interocular insertion. In some embodiments, the insertion is to the center of the eye. In some embodiments, the insertion is directly behind the cornea. In some embodiments, the insertion is directly inside the original crystalline capsular bag of the eye. In some embodiments, the insertion is about 17 mm from the retina. In some embodiments, the first lens is closer to the cornea. In some embodiments, the second lens is closer to the cornea.

In some embodiments, the lens systems of the invention are for use in correcting a defect in vision is a subject in need thereof. In some embodiments, the lens systems of the invention are for use in repairing damaged vision. In some embodiments, a defect or damage in vision is selected from: myopia, hyperopia, presbyopia, cataracts, macular degeneration, retinal neuropathy and glaucoma. In some embodiments, the defect in vision is myopia or presbyopia.

In some embodiments, the lens systems of the invention are for use in enhancing or amplifying vision. In some embodiments, the lens systems of the invention are for use in optical zooming. In some embodiments, the zoom is at least a 1.25×, 1.5×, 1.75×, 2×, 2.25×, 2.5×, 2.75× or 3× zoom. Each possibility represents a separate embodiment of the invention. In some embodiments, the zoom may be anywhere between a 1.25× and 3× zoom. Each possibility represents a separate embodiment of the invention. In some embodiments, the lens systems of the invention are capable of multiple zooms. In some embodiments, the zoom is a 1.25× zoom.

By another aspect there is provided a vision correction and enhancement system, comprising:
a. any one of the lens systems of the invention; and
b. at least one laser diode capable of producing laser or LED light at at-least one wavelength capable of tuning the reflectivity of at least one of the semiconducting materials.

In some embodiments, a system includes one or more laser diodes, LED lights or a combination thereof. In some embodiments, there are two diodes, two LED lights or one diode and one LED light. In some embodiments, the diode or LED light emits light at only one wavelength. In some embodiments, the diode or LED light emits light at the wavelength such that the emitted light tunes the reflectivity of the first or the second semiconducting material of the lens system. In some embodiments, the two diodes or LED lights emit light at different wavelengths. In some embodiments, the two different wavelengths are the wavelength to tune the first and the wavelength to tune the second semiconducting material of the lens system. In some embodiments, the diode or LED light emits light at multiple frequencies. In some embodiments, the diode or light is can be directed to emit light at a specified frequency. In some embodiments, the system includes only one diode or LED light that can be directed to emit light at the desired frequencies of the two semiconducting materials of the lens system. In some embodiments, the laser diode or LED is capable of producing external excitation light at a plurality of wavelengths capable of tuning the reflectivity of the first and second semiconducting materials.

As used herein, the term “diode” refers to a semiconductor device that produces coherent radiation when current passes through it. In some embodiments, the diode is a laser diode. In some embodiments, the diode is a light-emitting diode. In some embodiments, the radiation produced by the diode is visible light. In some embodiments, the light produced by the diode is infrared light. In some embodiments, the light produced by the diode is ultra violet light.

In some embodiments, the laser diode or LED is mounted on a device and configured to shine/project light on the lens system. In some embodiments, the device is glasses. In some embodiments, the device is a device that can be positioned near the eye. In some embodiments, device is a visor, cap, hat, glasses, goggles, monocle or other device that can be worn near the eyes. In some embodiments, the device is configured to block light at or near the desired wavelength that is not from the diode or LED. In some embodiments, the device is configured to block natural light at or near the desired wavelength. In some embodiments, the device comprises a filter that attenuates light at or near the desired wavelength. As used herein, “near” refers to within 50 nanometers (nm) of the desired wavelength. In some embodiments, the device is configured to block light below 400 nm. In some embodiments, the device and/or filter is configured to block blue light, and the tunable material is tunable by light within the blue spectrum. In some embodiments, the device is configured to limit light at or near the desired wavelength that is not from the diode or LED. In some embodiments, the laser diode or LED is mounted between a portion of the device that blocks light and the eye such that the device does not block the light from the laser diode or LED. In some embodiments, the mounting is configured such that the laser diode of LED is the primary source of light that may tune the reflectivity of the system of the invention.

In some embodiments, the device is a smartphone or other portable or handheld electronic device. In some embodiments, the device is a smartphone. In some embodiments, the laser diode or LED is in a smartphone. In some embodiments, the laser diode or LED is a component of the device. Examples of portable electronic devices include but are not limited to smartphones, tablets, music players, pagers, laptops and blue tooth ear pieces. In some embodiments, the laser diode or LED can be controlled by a smartphone or other computer. In such embodiments, a smartphone or device that includes a laser diode or LED may be held or operated such that the light from the laser diode or LED is projected on to the eye of the subject. In some embodiments, the laser diode or LED that is standard for a smartphone or device may be used as a component of the system.

In some embodiments, the vision correction and enhancement system of the invention are configured as a part of a vison correcting device. In some embodiments, the device is glasses, goggles or another form of eyewear. In some embodiments, the device further comprises a filter that blocks ambient light at or near the desired wavelength. Thus, the lens system of the invention can be imbedded in a piece of eyewear, such as goggles, with an internal light source in the eyewear that illuminates at the desired wavelength and with an optional filter on the outside of the goggles that would block light at or near the desired wavelength.

By another aspect there is provided a method of correcting and/or enhancing vision in a subject in need thereof, the method comprising inserting into an eye of said subject a lens system of the invention. In some embodiments, a lens system is inserted into each eye of a subject. In some embodiments, the method is for correcting vision. In some embodiments, the method is for enhancing vision.

In some embodiments, the inserting is performed during surgery. In some embodiments, the surgery is for a preexisting condition in the subject. In some embodiments, the inserting is performed during cataract or lens replacement surgery. In some embodiments, the methods of the invention further comprise providing the subject with at least one laser diode capable of producing laser or LED light at at least one wavelength capable of tuning the reflectivity of at least one of the semiconducting materials of the lens system. In some embodiments, the subject is provided the laser or LED mounted on a device such as is described herein.

In some embodiments, the method further comprises activating the laser diode or LED. In some embodiments, the method further comprises shining laser light and/or LED light at at least one wavelength capable of tuning the reflectivity of at least one of the semiconducting materials into the eye of the subject. In some embodiments, the method comprises tuning the reflectivity of the surfaces around the free space optical path to achieve vision enhancement. In some embodiments, the method comprises tuning the reflectivity of the surfaces around the lens to achieve vision correction. In some embodiments, vison enhancement and correction are achieved simultaneously by tuning the reflectivity of both sets of surfaces.

In the application, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their meanings in the art.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a surface” includes a plurality of such surfaces and reference to “the surface” includes reference to one or more surfaces and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### EXAMPLES

The following examples serve to illustrate certain embodiments and aspects of the present invention and are not to be construed as limiting the scope thereof. Many changes could be made in the specific embodiments disclosed herein while still obtaining an identical or similar result.

### Example 1: Tunable Materials

In order to achieve an improvement in the range and magnification of vision, an intraocular lens (IOL) whose reflectivity can be controlled, was designed with a schematic structure similar to that of FIG. 1. A lens system according to some embodiments of the invention may be referred to herein as SMARRT IOL or IOL. The multiple coatings in this IOL have tunable reflectivity as they are made from a semiconducting material whose reflectivity can be modified by illumination at a wavelength that is selectively absorbed by the semiconducting coating layer.

In some embodiments, a semiconducting coating made of Aluminum Nitride absorbs light at about 400 nm. For example, patient can wear sunglasses having an external coating that blocks the UV and the blue light illumination below 400 nm, such that bright light coming from the sun would be prevented from influencing the optical functionality of the IOL. A light source with a wavelength of 400 nm mounted on the same glasses and directed towards the IOL, would not be blocked by the filter of the external glasses, and hence initiate a change in reflectivity of the IOL. This light source can be triggered by the wireless Bluetooth or other transmission coming from a smart phone or it may be manually activated. In a controlled environment (e.g., indoors), the subject can remove the glasses and the illumination at 400 nm can come directly from a smart phone or other laser or LED light source.

In some embodiments, the spectral absorption and reflectivity of the semiconducting material used for coating surfaces as described herein may be enhanced or controlled by embedding, in the semiconducting material, nanoparticles through which light is selectively absorbed at an absorption peak in the infra-red (as can be designed using plasmonic resonance). In this way, the selectivity of an infrared wavelength is made such that the broad spectrum of ambient light from the sun (or other light sources) has a negligible effect over the reflectance coefficient of the layer. In some embodiments, the characteristics of the semiconducting material are such that the controlled light (coming from a smart phone, a source mounted on the spectacles or another location) coincides in wavelength with the absorption peak, e.g., maximal generation of free carriers is achieved, which enhances the layer's reflectivity.

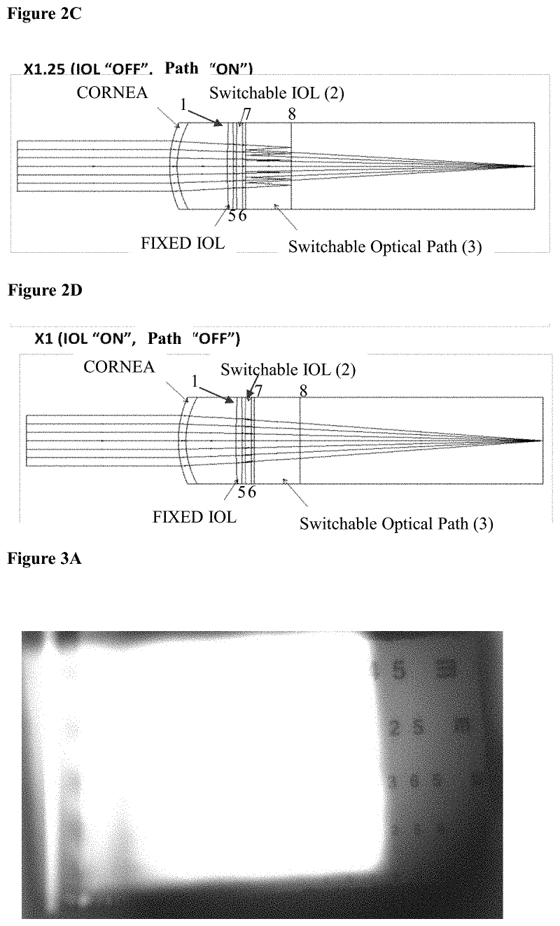

It should be noted that once the reflection coefficient of the semiconductor coating is modified by the laser signal, the increased reflectivity of the coating lasts only as long as the recombination time of the semiconductor, that is the time it takes to create or eliminate an electron or electron hole. Since this is a short time (in the range of micro seconds), the activation signal (laser illumination) should be present, or projected, onto the system for as long as the patient wishes the reflection to be increased. Also note that the semiconductor of the reflection coating around the lens (that increases the focal length) and the reflection coating around the free space distance Z (that yields Zₘ) can be made of different types of semiconducting materials, so that each one can be controlled and activated independently by two different wavelengths (e.g., coming from the external illumination source as described). In some embodiments, independent and simultaneous activation of reflective surfaces can be used to separate the multifocal and multi-zoom capabilities from each other with an appropriate lens element design, thereby giving the SMARRT IOL the possibility of titrating its specific sub-functions based on the specific needs of the cataract surgery patient. FIG. 2 depicts embodiments in which the sub-functions are operated independently. In some embodiments, simultaneous activation occurs by contacting the system with light that tunes both surfaces 5 and 6 and surfaces 7 and 8. In some embodiments, simultaneous activation comprises shining a single light source with a wavelength that excites surfaces 5, 6, 7 and 8. In some embodiments, simultaneous activation comprises shining a plurality of light sources with a plurality of wavelengths that excites surfaces 5, 6, 7, and 8. In some embodiments, surfaces 5 and 6 are excited by a different wavelength of light than surfaces 7 and 8. In such embodiments, simultaneous activation comprises shining light with at least two different wavelengths on the lens system.

### Example 2: Vision Correction

In some embodiments, the optical section of the SMARRT IOL includes two parts. The first part (lens assembly) is a combination of two lens elements: one outside the coated region and one inside it. The second part (optical path) is a bulk glass, or other non-reflective material, creating a free space optical path. This glass is within a coated region. Lens elements that are inside the coated region generate back reflections of the transmitted light (the visual image coming from an object) and thus the light passes several times through those coated lens elements before passing out of the IOL to its focal plane. Therefore, this SMARRT IOL achieves several focal lengths, such that a distance focus is achieved by light passage without any reflection (e.g., when m=1 and Zₘ equals the physical width of the bulk element), while intermediate and near focus is achieved by one or more back and forth internal reflections (e.g., with m=3, 5 and Zₘ equals 3 or 5 times the width of the lens element (Z)), thereby shortening the optical path length between the lens and the retina). In some embodiments, the SMARRT IOL achieves multiple zooming factors (e.g., no magnification when m=1 and Zₘ equals to the physical width of the element, but a fractional increase when m is 3 or more). Accordingly, embodiments of the invention include, enable and/or provide creation of a multifocal and multi-zooming IOL without the creation of unwanted aberrations or actively moving components that might decay and lose their efficacy over time.

The focal length and corresponding dioptric power of an IOL, as determined by the number of reflections m, can be expressed as:

$$\frac{1}{{f}_{m}} = \frac{1}{{f}_{0}} + \frac{m}{{f}_{a}} ⇒ {f}_{m} = \frac{{f}_{0} {f}_{a}}{{f}_{0} + {mf}_{0}} \qquad ( 2 )$$

For clinical trials, the reflectivity was chosen such that m is either 1 or 3 (since higher orders of reflection have irradiance losses that make them essentially negligible), and thus two prime focal lengths are realized. The optical path region, Zₘ, can be tuned due to reflectivity, since informational light passes several times through this region. The length of the optical path, Zₘ, as determined by the number of reflections m equals to:

Zₘ=mZ (3)

Here the reflectivity of the coating is such that the values of m are: m=1, 3, 5. As previously explained the combination of the various focal lengths, fₘ, in the lens assembly of the IOL, and optical paths, Zₘ, in the second part, yields an IOL with a multifocal and a multi-zoom lens capability.

### Example 3: Magnification

To describe the SMARRT IOL from an optical point of view, the IOL includes a lens with plurality of focal lengths fₘ which is located at a plurality of distances Zₘ from the retina. This property is optically equivalent to a set of focal planes fulfilling the imaging condition:

$$\frac{1}{u} + \frac{1}{{Z}_{m}} = \frac{1}{{f}_{m}} \qquad ( 4 )$$

and a set of magnifications (defined as the scale in the size of the object imaged on top of the retina) represented by equation:

M=Zₘ/u (5)

where u is the distance to the in-focus object. The set of focal lengths actually generates the capability of having several focal planes as the solution of u from Eq. 4, depending on m. In addition, several magnifications are possible from Eq. 5 where the magnification factor M depends on the free space distances Zₘ. Note that the magnification factor M is always less than one, so it actually minifies the object that is imaged on top of the retina. However, it is a magnification with respect to the image size of the object that would have been obtained without the addition of the device. The actual relative magnification M_{R} that an embodiment may provide in comparison to what would have been obtained without it equals to:

M_{R}=M/(17 mm/u)=Zₘ/17 mm (6)

where 17 mm is approximately the distance between the lens and the retina in a healthy eye.

Zemax is an optical design program used for the design and analysis of imaging and illumination systems. Some ZEMAX designs and results can be seen in FIGS. 2A-D. Reference is now made to FIG. 2. In the figure, the realization of multi focal capability (FIGS. 2A and 2B) and a zoom capability (FIGS. 2C and 2D) are demonstrated.

Specifically, in FIG. 2A use of a lens system according to some embodiments for looking at a faraway object is depicted. In some embodiments, surfaces coated with tunable materials are referred to as switchable plates. In FIG. 2A, switchable plates (which in some embodiments are around the second lens) are not engaged (for simplicity the free space optical path is not shown). In FIG. 2B use of a lens system according to some embodiments for looking at a near object is depicted. In some embodiments, switchable plates (which in some embodiments are around the second lens) are engaged (contacted with light at a wavelength that excites the material) and create back reflectivity as shown (for simplicity the free space optical path is not shown). As shown in FIG. 2B, the reflection increases the focal length and thus can correct for myopia or presbyopia. In some embodiments, a concave lens may be used to correct hyperopia, and other lens types may be inserted as necessary for proper vision correction.

In FIG. 2C use of a lens system according to some embodiments for magnification is shown. In some embodiments, the switchable plates (coated surfaces) around the second lens are not engaged (excited) and the switchable plates around the free space optical path are engaged and create a resonator. For example, by increasing the effective distance to the retina, the image is magnified by 1.25×. In FIG. 2D use of the lens system as in 2A is shown, but with the free space optical path present and the surfaces around that path not engaged as shown.

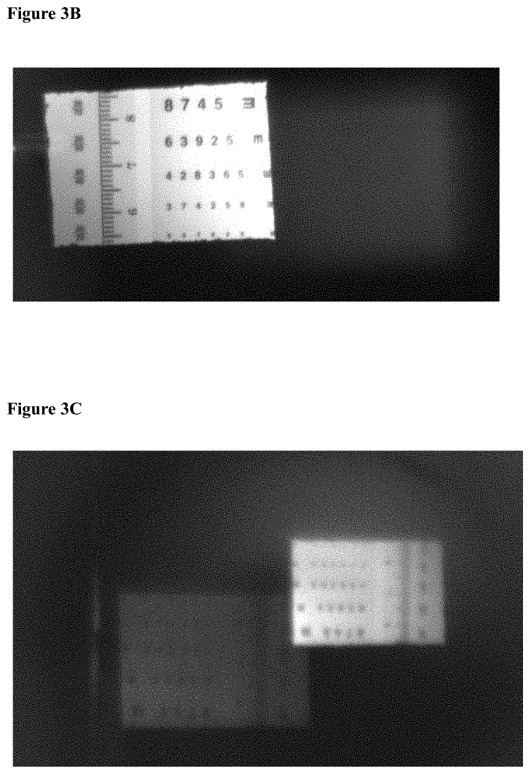

To demonstrate the bifocal capability of the IOL, two eye charts were placed side by side, one 80 cm from the IOL (Right side of FIGS. 3A and 3B) and one was 30 cm away (Left side of FIGS. 3A and 3B). With none of the tunable surfaces engaged the eye chart from 80 cm is in focus as can be seen in FIG. 3A. When the tunable surfaces around the lens 2 are excited, the image from 30 cm away comes into focus clearly as can be seen in FIG. 3B.

Similarly, a single eye chart was imaged using the zooming tool of the IOL as can be seen in FIG. 3C. Two images at different zooms (for 1 and 3 passes through the free space optical path) for the one object positioned at a fixed axial distance are produced.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A lens system comprising:
a first lens and a second lens arranged coaxially along a central axis, said central axis passing through vertices of said first and second lenses; and
a plurality of surfaces arranged along said central axis, wherein
(a) at least said second lens is sandwiched between two of said surfaces, and at least one of said surfaces being at least partially coated with a first semiconducting material with tunable-reflectivity; and
(b) at least two of said surfaces are at least partially coated with a second semiconducting material with tunable-reflectivity and define between them a free space optical path.

2. The lens system of claim 1, wherein
a. the distance between said first lens and said second lens is no more than 100 mm,
b. the free space optical path extends along the central axis between 1 and 100 mm, or
c. the first lens, second lens or both has a thickness of between 0.1 and 10 mm.

3. (canceled)

4. (canceled)

5. (canceled)

6. The lens system of claim 1, wherein said coating with a first semiconducting material with tunable-reflectivity faces said second lens.

7. The lens system of claim 1, wherein said coating with a second semiconducting material with tunable-reflectivity faces the interior of the free space optical path.

8. The lens system of claim 7, wherein light entering said free space optical path is reflected between coated surfaces of the path, optionally wherein said reflecting creates a resonator between the coated surfaces of the path.

9. (canceled)

10. The lens system of claim 1, wherein at least one of said first and second semiconducting materials is tunable by contact with a laser or LED light.

11. The lens system of claim 10, wherein said laser or LED light's wavelength is not greater than 400 nm, said first semiconducting material is tunable by contact with a first laser or LED light and said second semiconducting material is tunable by contact with a second laser or LED and wherein said first laser and said second laser have different wavelengths or both.

12. (canceled)

13. (canceled)

14. The lens system of claim 1, wherein said first or second semiconducting material is selected from the group consisting of: semiconductors absorbing at the desired wavelength, semiconductors with synthesized or engineered bandgaps allowing enhanced absorption at the desired wavelength, and a surface having plasmonic nanostructures to enhance the surface light absorption process at the desired wavelength.

15. The system of claim 1, wherein said first and second semiconducting materials with tunable-reflectivity are the same.

16. The system of claim 1, wherein said first or second semiconducting material is aluminum nitride.

17. The lens system of claim 1, configured for interocular insertion, optionally wherein said interocular insertion is about 17 mm from the retina.

18. (canceled)

19. (canceled)

20. (canceled)

21. (canceled)

22. (canceled)

23. The lens system of claim 1, further comprising a light source adapted to tune the reflectivity of at least one of the materials with tunable reflectivity, optionally wherein said light source is a laser or LED light.

24. (canceled)

25. A vision correction and enhancement system, comprising:
(a) the lens systems of claim 17; and
(b) at least one laser diode capable of producing laser or LED light at at-least one wavelength capable of tuning the reflectivity of at least one of the semiconducting materials.

26. The vision correction and enhancement system of claim 25, wherein said laser diode or LED is mounted on glasses and configured to shine laser light on said lens system, said laser diode or LED is capable of producing external excitation light at a plurality of wavelengths capable of tuning the reflectivity of said first and said second semiconducting materials, or both.

27. The vison correction and enhancement system of claim 26, wherein said glasses are configured to block light at or near the wavelength of the laser light produced by said laser diode or LED.

28. (canceled)

29. A method of correcting or enhancing vision in a subject in need thereof, the method comprising inserting into an eye of said subject the lens system of any-one-e claim 16.

30. (canceled)

31. The method of claim 29, further comprising providing to said subject at least one laser diode capable of producing laser or LED light at at-least one wavelength capable of tuning the reflectivity of at least one of the semiconducting materials of said lens system or shining into the eye of the subject laser light and/or LED light at at-least one wavelength capable of tuning the reflectivity of at least one of the semiconducting materials or said lens system.

32. (canceled)

33. The method of claim 31, for correcting vision wherein said laser light, LED light or both is at a wavelength capable of tuning the reflectivity of the surfaces around said second lens.

34. The method of claim 31, for enhancing vision wherein said enhancing is optical zooming, and wherein said laser light, LED light or both is at a wavelength capable of tuning the reflectivity of the surfaces around said free space optical path.
